(19) United States
(12) Patent Application Publication
(10) Pub. No.: US 2026/0137896 A1
(43) Pub. Date: May 21, 2026

(54) Hypoxic Mask with Adjustable Supplemental Air Intake

(71) Applicant: Hub Design LLC, Reno, NV (US)

(72) Inventor: David Greif, Reno, NV (US)

(21) Appl. No.: 19/382,970

(22) Filed: Nov. 7, 2025

Related U.S. Application Data
(60) Provisional application No. 63/722,749, filed on Nov. 20, 2024.

(51) Int. Cl. A61M 16/20; A61M 16/00; A61M 16/08; A61M 16/22

(52) U.S. Cl. CPC A61M 16/204; A61M 16/0003; A61M 16/0045; A61M 16/0875; A61M 16/22; A61M 2205/3303; A61M 2205/7509; A61M 2230/06; A61M 2230/205

(57) ABSTRACT

A breathing system that supplies air containing reduced oxygen levels includes a mask, a housing, a valve or other air flow restrictor, and one or more sensors that detect the amount of oxygen present in the system. A user breathes into the mask, after which the air is moved into the housing, and subsequently through an expandable reservoir after which the air can be re-breathed by the user. One or more sensors may be mounted to the user as well as contained within the housing. In the event that the amount of oxygen in the system's recirculated air falls below a predetermined level, the valve may be opened to allow ambient air containing higher levels of oxygen to enter the system. The system may contain a carbon dioxide scrub medium, as well as other filters to maintain desired characteristic of the air within the system.

### CROSS REFERENCE TO A RELATED APPLICATION

This application claims the benefit of provisional patent application U.S. App. No. 63/722,749, filed on Nov. 20, 2024 and entitled “HYPOXIC MASK WITH ADJUSTABLE SUPPLEMENTAL AIR INTAKE”, the entire contents of which are hereby expressly incorporated by reference into the present application.

### BACKGROUND AND SUMMARY

This invention relates to fitness and/or breathing products, and more particularly to a device that is used to simulate higher altitude conditions.

Many athletes, particularly competitive athletes, seek numerous ways to challenge their bodies by doing a variety of physical activities under different conditions. For instance, some athletes specifically train in high altitude locations, which results in a reduced number of oxygen molecules contained in the air. The reduced number of oxygen molecules in the air means that less oxygen is delivered to the user's body with each breath that is taken.

While individuals living or visiting locations at a high altitude are exposed to these conditions, there is a desire for individuals who are not living or visiting high altitude locations to be exposed to similar conditions. Additionally, individuals who are not athletically inclined may nevertheless desire to be exposed to these conditions prior to a trip to a high altitude location in order to acclimate one's body to the reduced oxygen intake.

In recent years, many athletes and other individuals have attempted to initiate intermittent hypoxia exposure (IHE), which consists of alternating periods of breathing of low oxygen air having characteristics similar to those at high altitudes, followed by periods of breathing ambient air wherever the user is located. Various studies have shown benefits of IHE.

The present invention provides a system that enables a user to easily initiate intermittent hypoxia exposure using a simple, portable device.

In one embodiment, a breathing system for supplying air containing reduced oxygen levels is provided. The system may include a mask, a housing connected to the mask, a reservoir connected to the housing, one or more sensors, including an oximeter sensor, and an air flow restrictor that enables air to enter the system in response to readings from within the breathing system. Additionally, the system may include oxygen sensors and carbon dioxide sensors.

The air flow restrictor varies flow rate of air into the system, and can be moved between multiple positions, including one where ambient air is permitted to enter the system, and another where ambient air is not permitted to enter the system. In one embodiment, the air flow restrictor may include a cap having a plurality of openings extending therethrough, and a flow pathway extending through a portion of the housing. When one of the plurality of openings and the flow pathway align, ambient air is permitted to enter the system. The air flow restrictor may be automatically or manually moved between the respective positions based on readings from one or more sensors, including the saturation of peripheral oxygen sensor, or based on a user's feedback.

The system may also include a carbon dioxide scrub medium that removes carbon dioxide from recirculated air, as well as other filters and dehumidifying/desiccants material or dehumidifying methods. The carbon dioxide scrub material, filters, and dehumidifying material may easily be removed from the system and replaced.

In another embodiment, a method of using a breathing system is provided. This may include the steps of selecting a target altitude or saturation of peripheral oxygen sensor setting, restricting entry of air into the breathing system by a supplemental air intake, such as a valve or other air flow restrictor, monitoring a saturation of peripheral oxygen sensor associated with a user, and adjusting the valve to control movement of air entering the system. Additionally, the method may include the step of monitoring various other sensors, such as oxygen sensors and carbon dioxide sensors associated with the system and/or the user. The method may also include the steps of breathing into a mask, moving air exhaled into the housing connected to the mask, and moving recirculated air from the housing into a reservoir connected to the housing. The air may be transported from the mask to a tube, after which, the air is transported from the tube though an inhalation port extending from the housing. The method may also include the steps of manually adjusting the valve by twisting a cap having a plurality of openings to align with a flow pathway extending through a portion of the housing, and varying the flow rate of ambient air into the system in response to readings from the user's saturation of peripheral oxygen sensor. Alternatively, the valve may automatically be adjusted based on readings from one or more of the sensors.

The invention also contemplates a method of using the system in order to initiate intermittent hypoxia exposure, substantially in accordance with the present application.

Other aspects, features and advantages of the invention will become apparent to those skilled in the art from the following detailed description and accompanying drawings. It should be understood, however, that the detailed description and specific examples, while indicating certain embodiments of the present invention, are given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements that can be seen in several views, and in which:

FIG. 1 is an isometric view of a system including a hypoxic mask with an adjustable supplemental air intake installed relative to a user, a reservoir, and an oximeter;

FIG. 2 is an exploded view of a main housing associated with the system of FIG. 1;

FIG. 3 is an isometric perspective view of the main housing of FIG. 2;

FIG. 4 is a sectional view of the main housing taken about line 4-4 of FIG. 3;

FIG. 5 is a top plan view of a filter and cap desiccant associated with the system of FIG. 1;

FIG. 6 is a sectional view of the filter assembly taken about line 6-6 of FIG. 5;

FIG. 7 is an isometric perspective view of a reservoir associated with the system of FIG. 1;

FIG. 8 is a sectional view of the main housing of FIG. 4 with arrows identifying a flow path when a user inhales;

FIG. 9 is a sectional view of the main housing of FIG. 4 with arrows identifying a flow path when a user exhales;

FIG. 10 is a sectional view of the main housing of FIG. 4 with arrows identifying a flow path when a user inhales with a flow valve being opened;

FIG. 11 is a sectional view of filter assembly of FIG. 6 showing a flow pattern;

FIG. 12 is a flow chart explaining use of the device;

FIG. 13 is a chart summarizing ideal gas concentrations in a closed system versus the present application system;

FIG. 14 is a chart summarizing ideal gas concentrations of the present application system;

FIG. 15. is an isometric view of another embodiment of the system including a mouthpiece.

In describing the representative embodiment of the invention that is illustrated in the drawings, specific terminology will be resorted to for the sake of clarity. However, it is not intended that the invention be limited to the specific terms so selected, and it is to be understood that each specific term includes all technical equivalents, which operate in a similar manner to accomplish a similar purpose. For example, the words “connected,” “attached,” or terms similar thereto are often used. They are not limited to direct connection but include connection through other elements where such connection would be recognized as being equivalent by those skilled in the art.

### DETAILED DESCRIPTION

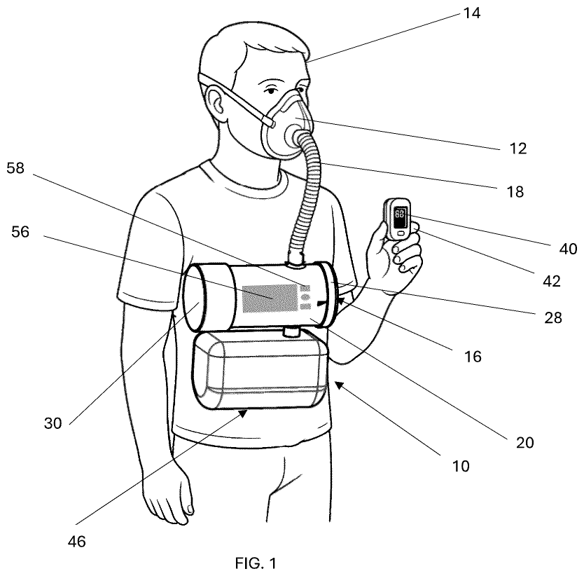

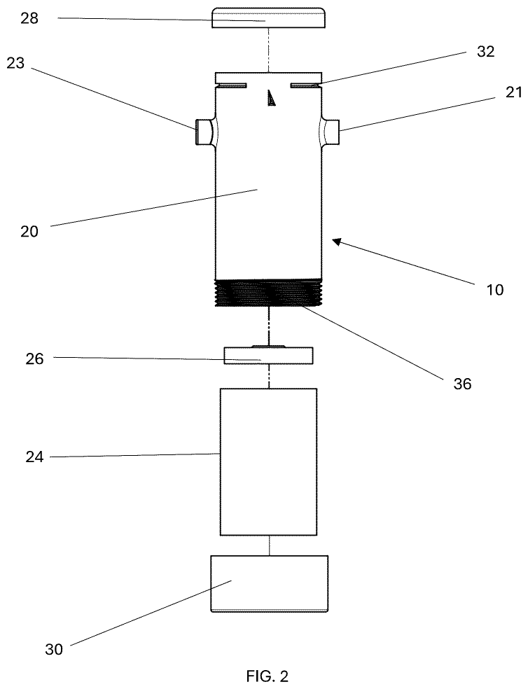

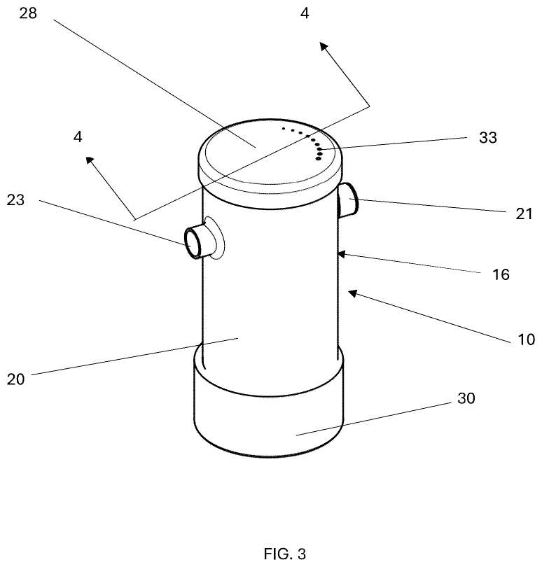

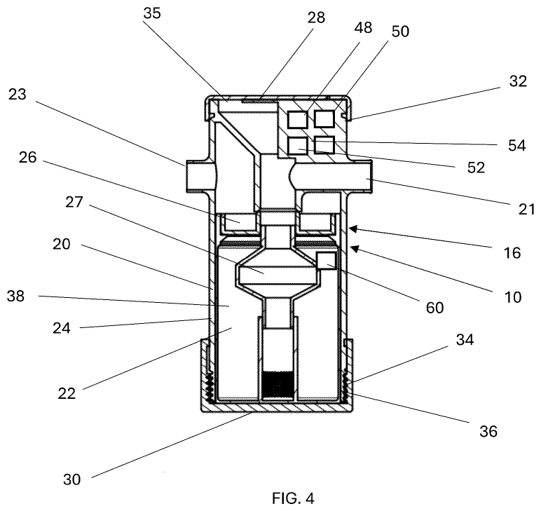

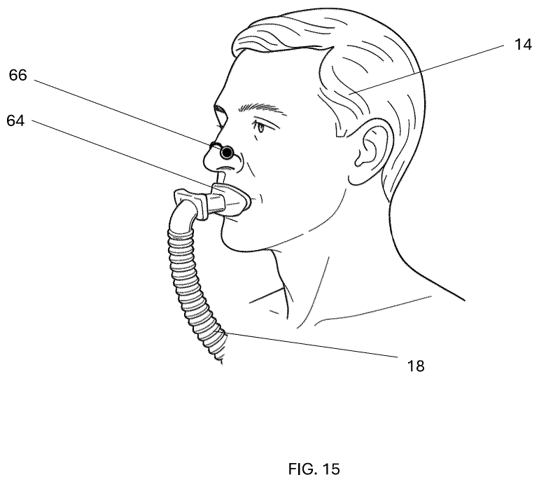

Referring to the figures, one embodiment of a system 10 that includes a portable mask 12 that is releasably affixable to a head 14 of a user to surround and cover the user's mouth and nose. While a mask 12 is shown in FIG. 1, the system 10 could similarly feature a mouthpiece 64 that engages with a user's mouth, as seen in FIG. 15. The system 10 is designed to imitate air conditions present at an elevation higher than the present altitude at a given location. More specifically, the system 10 allows users to recirculate a portion of air multiple times, as will be further described below, causing a reduction in the amount of oxygen contained within that air that is similar to air conditions at a higher elevation. This reduction in oxygen concentration during breathing creates a lower percentage of oxygen in blood saturation known as hypoxia. Hypoxia as well as the acclimation to this environment can be measured by a peripheral saturation of blood oxygen device (SpO₂), also referred to as a pulse oximeter 40. A typical adult undergoes hypoxia at an SpO₂ value below 90%. By monitoring a user's SpO₂ values during use and allowing the user to achieve a target SpO₂, the user will be able to simulate the reduced oxygen levels of a desired increased elevation. The body's response to hypoxia depends on the severity and duration of the hypoxia and subsequently red blood cell production. There is a direct correlation between an individual's red blood cell mass and maximal aerobic strength. This can be particularly beneficial for individuals including, but not limited to athletes or other individuals who want to improve their aerobic strength.

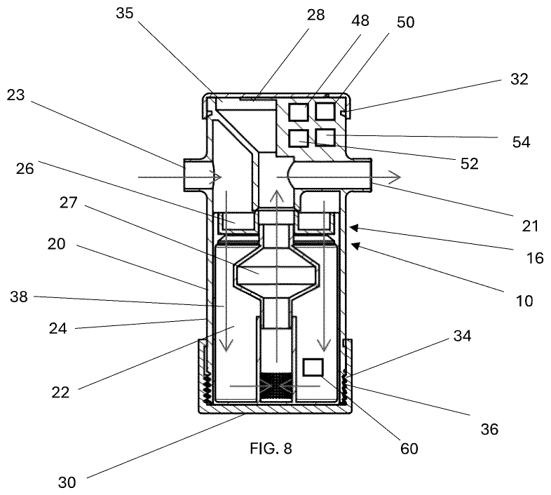

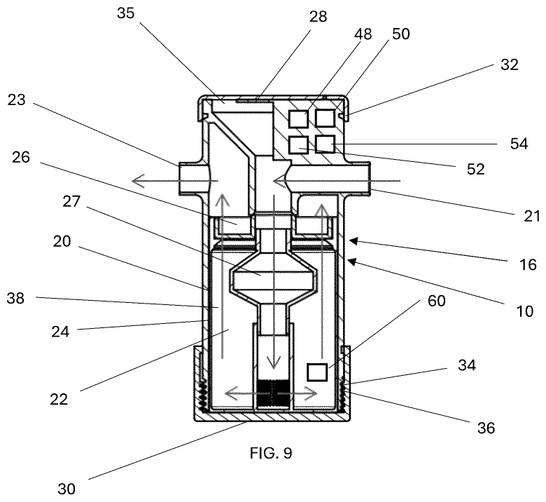

The mask 12 is attached to a housing 16 via a tube 18 or other delivery device. In an alternate embodiment, a mouthpiece 64 is used in conjunction with a nose clip 66 as seen in FIG. 15. While FIG. 1 shows the mask 12 connected to the tube 18 and other components, the mouthpiece could similarly be connected to the other components about the tube 18. The housing 16 is configured to receive air after a user exhales through the mask 12. See FIG. 9 After a user initially exhales through the mask 12, when a user subsequently inhales (See FIG. 8), air from the housing 16 is then delivered back through the tube 18 to the user. Air that is exhaled or inhaled will be collectively referred to as recirculated air herein. With each breath, the amount of oxygen contained in the recirculated air is reduced due to the oxygen that is absorbed by the user.

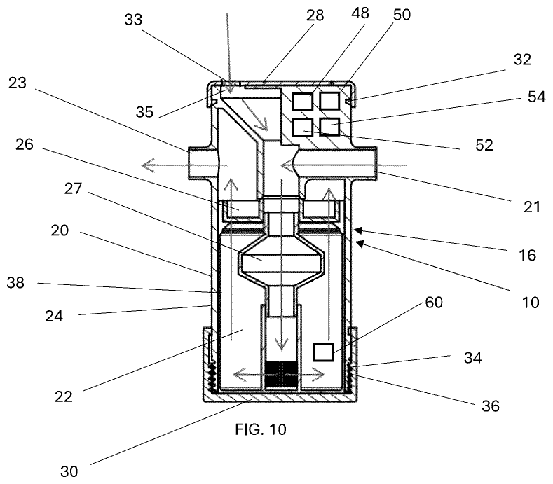

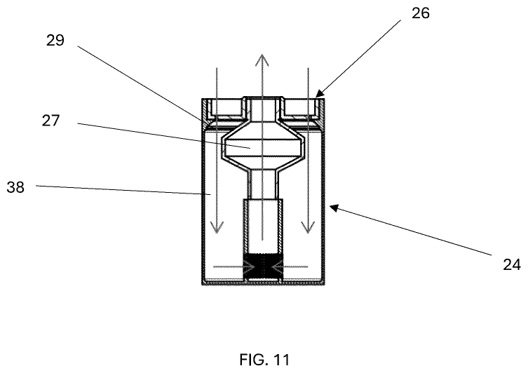

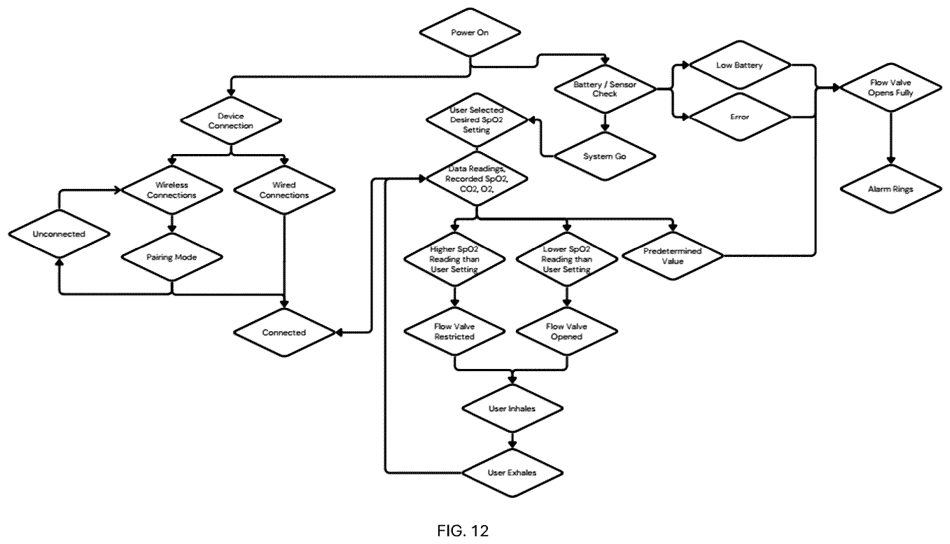

The housing 16 will now be further described. The housing 16 includes a main cannister 20 having an interior 22. The housing 16, main cannister 20, and/or interior 22 may include a variety of different air pathways, cavities, openings, and the like, some of which will be described herein. For instance, an inhalation port 21 and associated pathway extends through a side of the main cannister 20 to the interior 22, and an exhalation port 23 and associated pathway extends through an opposite side of the main cannister 20 to the interior 22. Of course, the direction of the ports could be inverted in certain embodiments. FIG. 8 shows the flow path when a user inhales, with air moving through the interior 22, and then out of the inhalation port 21. FIG. 9 shows the flow path when a user exhales, with air moving through the inhalation port 21 into the interior 22, and then out of the exhalation port 23. FIG. 10 shows another flow path when a user exhales, but when a supplemental air intake, such as a flow valve (described more below) is partially opened, with air moving through the inhalation port into the interior, with ambient air also entering the interior through the flow valve.

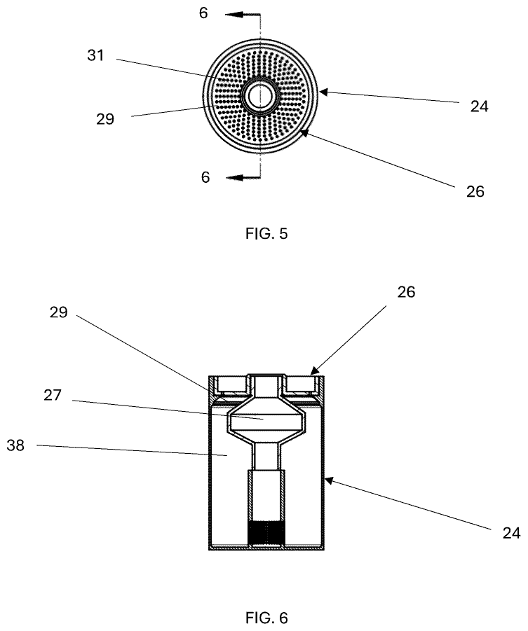

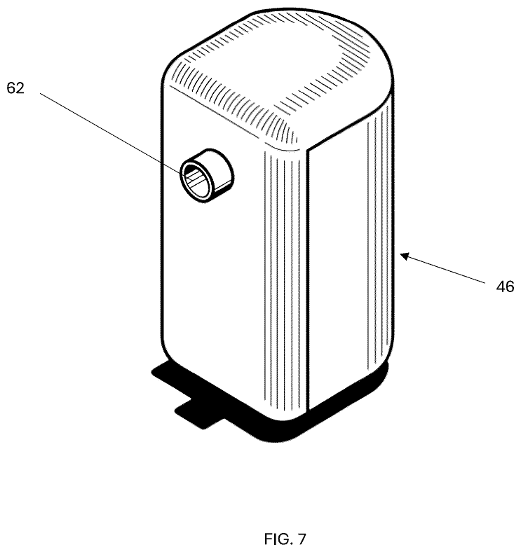

Turning to FIGS. 5 and 6, the housing 16 includes a filter housing 24 and a dehumidifying method, such as a cap desiccant 26, or other material or device that facilitates dehumidification, configured to be releasably inserted into the interior 22. The cap 26 is shown nested relative to the filter housing 24. The filter housing 24 may contain a filter insert 38 such as a carbon dioxide (CO₂) scrub. This could take the form of an existing medical-grade CO₂ scrub mediums like soda lime comprised of calcium hydroxide (Ca(OH)₂) and sodium hydroxide (NaOH). The carbon dioxide scrub medium filter insert 38 absorbs carbon dioxide that is present in the recirculated air. The typical absorption capacity rate of soda lime is 260 to 400 mL of CO₂ per gram and requires 0.06 to 0.09 grams per breath. The CO₂ scrub filter insert 38 can be designed and implemented into the filter housing 24 as a replaceable and exchangeable filter in the event when the CO₂ scrub medium has saturated and needs to be replaced. The filter housing 24 may also include a viral filter 27. The top 29 of the filter housing 24 may have a plurality of small perforated holes 31 that hold the scrub 38 and other components in place while allowing air to pass therethrough.

Additionally, the housing 16 includes one or more caps that engage with the main cannister 20 when the system 10 is in use. More specifically, a first cap 28 that serves as an air adjustable cap is releasably secured to the housing 16 at a first end. The first cap 28 may have a plurality of openings 33 formed therein having different sizes. As shown, the first cap 28 is secured to the main cannister using a snap fit type connection with slots 32 formed in the main canister 20. Thereafter, the first cap 28 is rotatable about the slots 32. A flow pathway 35 extends through a top side of the housing 16 and abuts the first cap 28. The flow pathway 35 and the openings 33 work together to create an air flow restrictor/flow valve as described above. More specifically, a user can twist the first cap 28 to align one of the openings 33 with the flow pathway 35 to allow ambient air to flow into the housing 16. For instance, depending on the oxygen levels detected in the system 10, a user may determine that additional oxygen is needed, in which case the first cap 28 is twisted to align one of the openings 33 with the flow pathway 35. Initially, the user may align a smaller opening 33 with the flow pathway 35. If additional oxygen is needed in the system, the user may align a larger opening 33 with the flow pathway 35. Once a desired oxygen level is reached, the user may leave the flow valve setting to maintain an equilibrium whereas the oxygen contained in the ambient air entering the system is proportional to the oxygen utilized by the user.

In alternate embodiments, the system 10 may also include a one-way valve (not shown) or other intake that controls the introduction of ambient air into the system 10 and subsequently a release valve to control expelled air. In certain embodiments, the air need not be ambient and instead could be supplied by a compressed oxygen cartridge (not shown) or any other air supply having oxygen levels that exceed the oxygen levels of recirculated air within the system 10. The valve could be any type of valve, such as ball valve, stopcock valve, or similar valve, that may be mounted to the tube 18, the housing 16 or another portion of the system 10. In the event that the various sensors detect that the oxygen levels fall below a desired threshold, or that other predetermined thresholds are being surpassed, the valve can be adjusted either manually or automatically via a motor such as a servo motor, or any other device that manipulates a valve, to introduce air with higher oxygen into the system 10, such as ambient air, oxygen from a compressed oxygen cartridge, or any other air supply. This enables prolonged use of the system 10 while maintaining safe levels of oxygen.

Additionally, a second cap 30 is releasably secured to the housing 16 at a second end. The second cap 30 may secure the filter housing 24 and cap desiccants 26 within the interior 22 of the main cannister 20. As shown, the second cap 30 is secured to the main cannister 20 using threaded surfaces 34 that engage with complimentary threaded surfaces 36 associated with the main cannister 20. Of course, the caps 28, 30 may be releasably engaged with the main cannister 20 using any other fasteners or securement mechanisms known in the art.

The system 10 also may include sensors that monitor the saturation of oxygen contained in the user's blood. As shown, this may be in the form of a monitor 40, such as a pulse oximeter, that may be mounted to a user's finger 42. Alternatively, other monitors (not shown) that may be mounted to a user's ear, a user's wrist, or an alternative location. The system 10 may include communication hardware 48 that transmits information and readings to a smartphone, tablet, computer, and the like (not shown). As will further be discussed below, the system 10 can make adjustments through the use of pneumatic controls or by manual adjustment based on the readings from the monitor 40, or any of the other monitors referenced herein. Additionally, the system includes a battery 50 that powers the various functions described herein.

The system 10 may include various additional gaseous sensors to monitor the conditions of the air. For instance, in the illustrated embodiment, at least one oxygen sensor 52 and at least one carbon dioxide sensor 54 may be located within the housing 16. This enables the system 10 and a user to understand the amount of oxygen and carbon dioxide that is present in the air within the housing 16 after each subsequent breath. By recording and collecting sensory data, the system 10 may assess a user's progress in acclimating to a higher altitude with an oxygen-deficient environment. Outside of system 10, acclimation to a reduced-oxygen environment is typically characterized by a gradual increase in an individual's SpO₂ value to a normoxic range of 97%-99% within 1-3 weeks of exposure to a higher altitude. By correlating the oxygen concentration in the recirculating air to the user's SpO₂ values, the system 10 can record and assess these values over multiple sessions to evaluate the user's acclimation to said environment and additionally equate the environment of system 10 to that of a higher elevation equivalent. In the event that the oxygen levels are too low or the carbon dioxide levels are too high, the system 10 can initiate an alarm to notify the user. The flow valve comprising the openings 33 and the flow pathway 35 may also be automatically opened when such unsafe or unwanted conditions are reached.

Additionally, the system 10 can track the absorption capacity of the CO₂ filter to alarm the user when a new filter is necessary. The system 10 can measure and evaluate the decreasing CO₂ absorption rate of the CO₂ filter per breath over time. When this rate reaches a predetermined value, an alarm may be triggered, otherwise, the flow valve can automatically be opened. The housing 16 may also contain wireless communication hardware 48, (e.g. Wifi, Bluetooth, etc.), as well as, wired communication (not shown) (e.g. USB, data ports, etc.) that transmits information relating to the operation of the system 10, such as the oxygen levels, carbon dioxide levels, blood oxygen saturation, usage metrics, and the like to external devices such as smartphones, tablets, computers, and the like. The housing 16 may also feature a rechargeable battery 50 or direct power supply (not shown). The housing 16 may also feature a display 56 for visualizing the feedback from such sensors of the system 10 including battery life, user's peripheral saturation of blood oxygen readings, and the recirculating air conditions within system 10. The housing 16 may also feature buttons 58 to adjust the desired peripheral saturation of blood oxygen levels and subsequently the desired altitude simulated by the device.

The system 10 may also include various components that adjust the conditions of the recirculated air. For instance, as described above, one or more air filters 27 may be provided within the housing 16 or elsewhere that filters toxins, viruses, and other impurities from the recirculated air before it is returned to the mask 12. The air filter 27 is designed to prevent particulates from the CO₂ filtration medium to be inhaled by the user, as well as prevent the spread of airborne illness while also maintaining a low airflow resistance. The lifespan of the filtration should adequately match or exceed the lifespan of the CO₂ filtration. Additionally, a dehumidifier 60 may be enclosed within the housing 16 or elsewhere to reduce the moisture present in the recirculating air. A non-limiting example of dehumidifying techniques include disposable silicon dioxide, clay, calcium chloride desiccants, and the like. A non-limiting example of active dehumidifying techniques includes thermoelectric, desiccant wheels, electrolytic, and the like.

Additionally, the system 10 includes a reservoir 46, for instance an expandable or flexible reservoir, and as shown an exhaust bladder that holds exhausted air after the air travels through the housing 16. When the user exhales, the exhausted, oxygen-depleted air travels through the tube 18, through the housing 16 as shown in FIG. 9, and is then transported through the exhalation port 23, and through a reservoir port 62 and associated pathway, where it is then captured within the reservoir 46. The size of the reservoir 46 is, at a minimum, capable of holding the volume of exhausted air. This volume is related to an individual's lung capacity and during tidal breathing can range from 500 milliliters to 3 liters for an average adult. When the user subsequently inhales, the oxygen-depleted, recirculated air contained within reservoir 46 travels in a reverse direction through the reservoir port 62, through the exhalation port 23, through the housing 16 as shown in FIG. 8, through the inhalation port 21, and then through the tube 18 and mask 12 for inhalation. This flow of air repeats for each breath cycle, and the recirculated air is mixed with ambient air via the flow valve formed by the combination of the flow pathway 35 and the openings 33, as needed, to ensure the user's blood oxygen content remains at a desired level.

The system 10 may include a release valve (not shown) which may be located on or around the reservoir 46 of the reservoir port 21, such as a one-way exhaust valve. The release valve allows some of the recirculated air to exit the system 10 such that the volume of air within the capacity of the reservoir 46 is not exceeded. For instance, the exhaust valve may be a low-pressure release valve designed to open when the reservoir 46 reaches capacity during the end of the exhalation phase of the user's breathing and is designed to not interfere with the user's comfort or require over-exertion in exhalation. For example the release-pressure could be akin to 900 pascals or less, which is an accepted comfort pressure level during tidal breathing.

The system 10 may additionally communicate and interact with an application loaded on the smartphone, tablet, computer, and the like. Such an application enables a user to monitor and evaluate use of the system 10 and all associated sensors including physiological responses over time. For instance, the application can collect and organize relevant metrics associated with use of the system 10, including duration of use, characteristics of the air including oxygen and carbon dioxide levels, saturation of peripheral oxygen levels, heart rate, assess acclimation progress, and the like. Under the access acclimation progress, based on the inputs, the application associated computes one or more physiological parameters, including but not limited to estimated oxygen uptake (VO₂), carbon-dioxide production (VCO₂), respiratory exchange ratio (RER=VCO₂/VO₂), ventilatory equivalents (VE/VO₂ and VE/VCO₂), end-tidal CO₂ (PETCO₂) and end-tidal O₂ (PETO₂), and an estimate of peripheral oxygen extraction. The system further stores time-stamped session data and executes algorithms to determine changes in the foregoing parameters across multiple sessions. From these changes, the application generates an acclimation metric or score indicative of a user's adaptation to hypoxic exposure. In various embodiments, the acclimation metric is computed from a weighted combination of (i) improvements in SpO₂ responses during standardized hypoxic bouts, (ii) changes in PETCO₂ and PETO₂ at rest or submaximal exercise, and/or (iii) increases in estimated oxygen uptake or peripheral extraction. The system may present the acclimation metric and underlying parameter

The system 10 can additionally provide programmed, timed intervals with recommended target SpO₂ levels or system oxygen levels for the desired simulated altitude for the user during use. The application can also link to other devices, such as smart watches and other devices or applications that measure and quantify physical fitness activities. The oxygen monitor 40 may also be employed, either via direct communication with a valve or via indirect communication with the valve through the associated application or a message to a user to adjust the cap 28 and associated openings 33 relative to the flow pathway 35, to control the position of the valve in order to allow introduction of sufficient ambient air to maintain the user's blood oxygen content at a desired level.

In yet another embodiment that is not illustrated, the system 10 may have the various components described herein with the exception of the housing. In this embodiment, a user breathes into a mouthpiece that is connected to a tube, and the tube is connected to a carbon dioxide scrub material. A particulate and viral filter may be mounted within the tube. The carbon dioxide scrub material is attached to an expandable bag reservoir configured to receive air after it is exhaled and travels through the filter and the carbon dioxide scrub material. An adjustable valve is located adjacent to the expandable bag reservoir that can be adjusted to enable intake of ambient air into the system. Again, a blood oxygen saturation measuring device may be mounted to the user, for instance on the user's finger.

FIG. 12 provides a flow chart of use, including the system operations associated with using the device. A summary follows, although it should be noted that the system does not necessarily require every described step, the system may include additional steps not shown or described, and the various steps may occur in orders differently than they are shown and described herein. As can be seen, initially, the system is powered on. Next, the system may connect to a device, otherwise, the battery and sensors are checked. If the system connects to a device, either a wireless or wired connection is created. If using a wireless connection, a pairing mode is initiated, which will repeat until pairing is successful. Once connected, data readings are recorded, including for instance SpO₂, CO₂, and O₂ readings. Where data readings and recordings are described herein in connection to SpO₂, oximeter, or oxygen, it should be understood that these traditionally are measured or detected using sensors/monitors mounted to a user's finger, whereas when the phrase O₂ is used herein, this is in connection to the amount of oxygen present in the air.

If the system is not connected to a device, the battery and sensors are checked. If the battery is low, a low battery message is delivered, and if any of the sensors are not properly working, an error message is delivered. In either or both events, the flow valve may open fully and/or an alarm rings.

If the system is connected to a device, the battery and sensors are checked, and if the battery has sufficient power and the sensors are properly operational, the system is ready to begin operation, and a user selects a desired SpO₂ setting. Next, data readings are recorded, including for instance SpO₂, carbon dioxide, and oxygen readings.

After the initial data recordings are completed, the system may detect a higher SpO₂ reading than the user setting, a lower SpO₂ reading than the user setting, or a predetermined value. If the predetermined value is reached, the flow valve is fully opened and/or an alarm rings. If the SpO₂ reading is higher than a user setting, the flow valve is restricted, whereas if the SpO₂ reading is lower than a user setting, the flow valve is opened. Thereafter, a user inhales, then exhales, and the data readings may repeat.

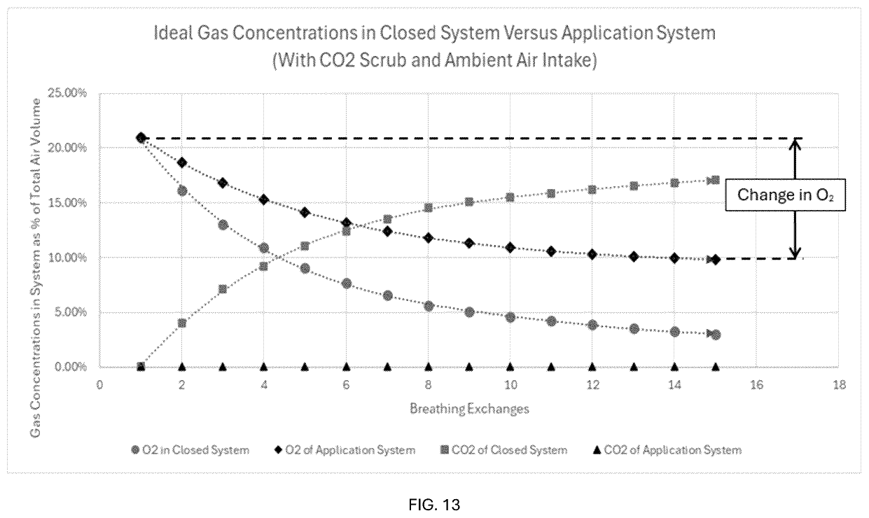

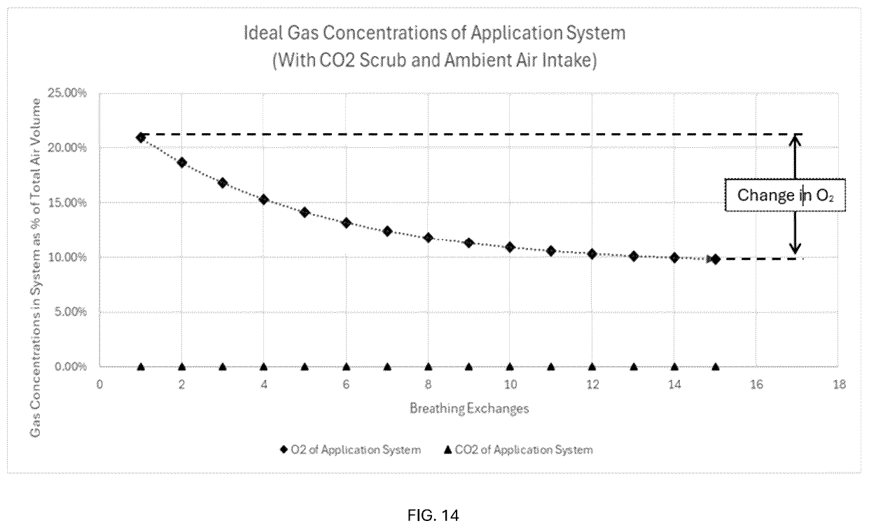

Finally, FIG. 13 shows a chart that visualize the reduction in oxygen in the system and increased carbon dioxide concentrations in the system 10 during recirculatory breathing compared to a closed system. FIG. 14 shows a similar chart only documenting the levels in the proposed system 10 in isolation. The circle markers relate to the oxygen levels and the square markers relate to the carbon dioxide levels of a closed system that does not include a valve or intake like the disclosed system. The diamond markers relate to the oxygen levels and the triangles markers relate to the carbon dioxide levels associated with the system 10 described above, with the carbon dioxide scrubber feature. FIG. 14 identifies the change in oxygen levels. As can be seen, the change in values of carbon dioxide reflect the ideal gas exchanges with the ambient air intake versus a completely closed system with no ambient air intake or carbon dioxide scrub. This helps demonstrate how ambient air intake impacts the rate of change of oxygen in the system, as well as the change in oxygen levels from an ambient start position to stable levels that represent the change in altitude while inducing hypoxia. The carbon dioxide levels remain at zero because of the carbon dioxide scrub material. Of course, the amount of ambient flow allowed into the system dictates the change in oxygen levels.

Although the best mode contemplated by the inventor of carrying out the present invention is disclosed above, practice of the present invention is not limited thereto. It will be made clear that various additions, modifications and rearrangements of the aspects and features of the present invention may be made in addition to those described above without deviating from the spirit and scope of the underlying inventive concept. The scope of some of these changes is discussed above. The scope of other changes to the described embodiments that fall within the present invention but that are not specifically discussed above will become apparent from the appended claims and other attachments. It is also understood that the invention disclosed and defined herein extends to all alternative combinations of two or more of the individual features mentioned or evident from the text and/or drawings. All of these different combinations constitute various alternative aspects of the present invention.

I claim:

1. A breathing system for supplying air containing reduced oxygen levels comprising:
a mask;
a housing connected to the mask;
a reservoir connected to the housing;
an oximeter sensor; and
an air flow restrictor that enables air to enter the housing in response to readings from within the breathing system.

2. The breathing system of claim 1, further comprising:
an oxygen sensor located within the housing; and
a carbon dioxide sensor located within the housing.

3. The breathing system of claim 1, wherein the air flow restrictor varies flow rate into the housing; and
wherein the air flow restrictor is movable between:
a first position in which flow of air into the housing is restricted; and
a second position in which flow of air into the housing is enabled.

4. The breathing system of claim 3, wherein the air flow restrictor further comprises:
a cap having at least one opening extending therethrough; and
a flow pathway extending through a portion of the housing;
wherein the at least one opening aligns with the flow pathway in the second position.

5. The breathing system of claim 3, wherein the air flow restrictor is moveable between the first position and the second position in response to readings from a saturation of peripheral oxygen sensor.

6. The breathing system of claim 3, wherein the air flow restrictor varies flow of ambient air into the system.

7. The breathing system of claim 1, further comprising a carbon dioxide scrub medium that removes carbon dioxide from recirculated air.

8. A method of using a breathing system comprising the steps of:
selecting a desired oxygen level or saturation of peripheral oxygen sensor setting;
restricting entry of air into the breathing system by a valve;
monitoring a saturation of peripheral oxygen sensor associated with a user; and
adjusting the valve to control movement of air entering the system.

9. The method of claim 8, further comprising the step of monitoring oxygen and carbon dioxide levels of recirculated air that is breathed into the system.

10. The method of claim 8, further comprising the steps of:
breathing into a mask;
moving air exhaled into the mask into a housing connected to the mask; and
moving recirculated air from the housing into a reservoir connected to the housing.

11. The method of claim 10, further comprising the steps of:
transporting air from the mask to a tube;
transporting air from the tube through an inhalation port pathway extending through the housing; and
moving air through the housing.

12. The method of claim 10, further comprising the steps of:
manually adjusting the valve by twisting a cap having at least one opening extending therethrough to align with a flow pathway extending through a portion of the housing; and
varying the flow rate of ambient air into the system in response to readings from the user's saturation of peripheral oxygen sensor.

13. The method of claim 8, further comprising the step of automatically adjusting the valve to vary the flow rate of ambient air into the system in response to readings from the user's saturation of peripheral oxygen sensor or another system sensor.

14. The method of claim 8, further comprising the step of removing carbon dioxide from recirculated air using a carbon dioxide scrub.

15. The method of claim 14, further comprising the steps of:
monitoring the conditions of the carbon dioxide scrub;
removing the carbon dioxide scrub once the carbon dioxide scrub reaches a saturation of lifespan; and
removably securing a second carbon dioxide scrub within the system.

16. The method of claim 8, further comprising the steps of moving recirculated air through a viral filter; and
dehumidifying the recirculated air.

17. The method of claim 8, further comprising the step of collecting metrics associated with one or more of duration of use, characteristics of the air, saturation of peripheral oxygen levels, heart rate, and assess acclimation progress.

18. A breathing system for supplying air containing reduced oxygen levels to a user comprising:
a mask;
a housing comprising:
a main cannister;
an interior;
an inhalation port extending from the main cannister in airflow connection to the mask; and
an exhalation port extending from the main cannister
a reservoir in airflow connection to the exhalation port of the housing;
at least one sensor; and
an air flow restrictor that enables air to enter the housing in response to readings from the at least one sensor.

19. The breathing system of claim 18, wherein the air flow restrictor further comprises:
a flow pathway extending through the main cannister;
at least one opening formed in a first cap that is movably mounted to the main cannister;
wherein the first cap is movable between:
a first position where the flow pathway aligns with at least one of the at least one opening to enable airflow into the main cannister; and
a second position where the flow pathway does not align with the at least one opening to inhibit airflow into the main cannister.

20. The breathing system of claim 18, further comprising a carbon dioxide scrub medium releasably insertable into the interior, wherein the carbon dioxide scrub medium is configured to remove carbon dioxide from recirculated air.
